(19) United States
(12) Patent Application Publication
(10) Pub. No.: US 2019/0083437 A1
(43) Pub. Date: Mar. 21, 2019

(54) S- Methylcysteine Sulfoxide For Prostate Cancer Prevention And Treatment

(71) Applicant: QUADRAM INSTITUTE BIOSCIENCE, Norwich, Norfolk (GB)

(72) Inventors: Richard Mithen, Norwich (GB); Antonietta Melchini, Norwich (GB); Maria Traka, Norwich (GB)

(21) Appl. No.: 16/087,964

(22) PCT Filed: Mar. 24, 2017
(86) PCT No.: PCT/GB2017/050838

§ 371 Date: Sep. 24, 2018

(30) Foreign Application Priority Data
Mar. 24, 2016 (GB) 1605013.0

(51) Int. Cl. A61K 31/198; A61K 31/7028; A61K 36/31; A61K 36/88; A61P 35/00; A23L 33/10; A23L 23/00

(52) U.S. Cl. CPC A61K 31/198; A61K 31/7028; A61K 36/31; A23V 2002/00; A61P 35/00; A23L 33/10; A23L 23/00; A61K 36/88

(57) ABSTRACT

The present invention relates to S-methylcysteine sulfoxide and compositions thereof for use in the treatment or prevention of prostate cancer. It also relates to analogues, derivatives or metabolites of S-methylcysteine sulfoxide for use in the treatment or prevention of prostate cancer. Compositions of S-methylcysteine sulfoxide or its analogues, derivatives or metabolites may be provided for oral consumption, for example as a pharmaceutical composition, a medicament or a food composition.

### TECHNICAL FIELD OF THE INVENTION

The disclosure herein relates to S-methylcysteine sulfoxide and compositions thereof for use in the treatment or prevention of prostate cancer.

### BACKGROUND OF THE INVENTION

Prostate cancer is one of the most common cancers in men, the symptoms of which may only become apparent after a number of years. There are different stages of prostate cancer: localised prostate cancer (e.g. early stage prostate cancer), locally advanced prostate cancer and relapsed and metastatic prostate cancer. Survival mainly depends on early diagnosis of the cancer. Early stage prostate cancer can be diagnosed by prostate-specific antigen (PSA) testing and/or digital rectal examination or biopsy. Later stage prostate cancer may require further tests such as magnetic resonance imaging (MRI), computerised tomography (CT) scan and/or isotope bone scan. The current treatment options include radical prostatectomy (surgery to remove the prostate), radical radiotherapy (radiation treatment to destroy the cancer cells), hormone therapy (drugs that reduce or stop the production or block the effect of hormones that help cancer cells to grow), brachytherapy (radiation treatment directed at the cancer from inside the prostate), pelvic radiotherapy (radiation treatment for men with locally advanced prostate cancer—destroys cancer cells that have spread outside the prostate) and orchidectomy (surgery for men with metastatic prostate cancer) (http://www.nice.org.uk/).

There is however a need for improved methods for treating early stage prostate cancer and/or providing prophylactic treatments to reduce cancer occurrence. Of men diagnosed with localised prostate cancer about one third will progress to advanced disease but it is not possible to identify which men will progress. Currently, there are no therapeutic treatments for men with localised prostate cancer apart from surgery to remove the prostate gland. There is an increasing risk of the cost and potential negative consequences of surgical intervention at this stage. Additionally, men with localised prostate cancer are often advised to participate in a programme of ‘Active Surveillance’ in which the progress of their prostate cancer is monitored through changes in plasma PSA levels, analysis of biopsy tissue or through MRI. Therefore, there is a need to develop dietary or other therapeutic means to reduce the probability of prostate cancer progression that could be offered to men on an active surveillance programme.

In addition, for all men there is a life-time risk of 1 in 8 in being diagnosed with prostate cancer. The risk rises substantially after the age of 50, and is also greater for men with an African genetic heritage and/or have a father or brother who has been diagnosed with prostate cancer or a mother or sister with breast cancer. Thus, a prophylactic means to reduce prostate cancer through easy to adopt therapeutic or dietary solution would be of great value for example to those of increased risk.

### SUMMARY OF THE INVENTION

Epidemiological studies have suggested that diets rich in cruciferous vegetables can both prevent prostate cancer and reduce the risk of progression from localised to advanced prostate cancer (see, for example, Steinbrecher et al. Int J Cancer. 2009 Nov. 1; 125(9):2179-86, “Dietary glucosinolate intake and risk of prostate cancer in the EPIC-Heidelberg cohort study” and Richman, E. L. et al. (2012) Vegetable and fruit intake after diagnosis and risk of prostate cancer progression. Int. J. Cancer 131, 201-210).

The present inventors have identified a component of broccoli, S-methylcysteine sulfoxide (3-(methylsulfinyl)alanine; SMCSO; methiin) and demonstrated an accumulation of inorganic sulphate in the prostate whilst the level of ADP increases. In particular, the present inventors have demonstrated an accumulation of SMCSO in prostate tissue, and an accumulation of sulfate and ADP. Whilst not wishing to be bound by any theory, the inventors have established that SMCSO can deplete ATP in cells suggesting a mechanism for reducing cancer cells. Accordingly, the present invention relates to S-methylcysteine sulfoxide (3-(methylsulfinyl)alanine; SMCSO) for use in the treatment or prevention of prostate cancer. The invention therefore also relates to providing SMCSO as a medicament or food composition for use in the treatment or prevention of prostate cancer. The invention further provides using SMCSO in combination with other components from high glucosinolate cruciferous vegetables, or high SMCSO-containing vegetables.

### DETAILED DESCRIPTION OF THE INVENTION

S-Methylcysteine sulfoxide is found in a number of vegetables, in particular in Brassica vegetables (Edmands W. M. B., et al. S-Methyl-L-cysteine sulphoxide: the Cinderella phytochemical? Toxicol Res 2, 11-22 (2013)).

A study in healthy male volunteers revealed that S-methylcysteine sulfoxide degrades to sulfate (SO₄²⁻) and that urine was the major route of excretion (Waring R. H. et al. Degradation to sulphate of S-methyl-L-cysteine sulphoxide and S-carboxymethyl-L-cysteine sulphoxide in man. Drug Metabolism and Drug Interactions 19(4), 241-255 (2003)). The present inventors have shown that inorganic sulphate occurs at very low levels in prostate biopsy tissue of men with or without prostate cancer, but accumulates in prostate tissue following a 12 month diet of a broccoli soup that is rich in S-methylcysteine sulfoxide (FIG. 3). The present inventors propose that the sulfate accumulation is a biomarker of the exposure of prostate tissue to S-methylcysteine sulfoxide through the biochemical process outlined in FIG. 1. S-Methylcysteine sulfoxide is metabolised by two major routes as described, for example, by Edmands et al. Identification of urinary biomarkers of cruciferous vegetables consumption by metabolic profiling Journal of Proteomic Research 10, 4513-4521 (2011) and Waring et al. Firstly, cysteine β lyase activity may cleave the molecule to generate pyruvate and sulfate. Secondly, aminotransferase activity may remove the amino group to generate 3-methylthiopyruvic acid sulfoxide. Associated with these metabolic routes is a depletion of ATP.

Exposure of prostate tissues to S-methylcysteine sulfoxide may occur either through the systemic circulation in the blood stream, or through urinary reflux. Indeed, urinary reflux into prostatic ducts is common knowledge in the field (Kirby R. S. et al. Intra-prostatic urinary reflux: an aetiological factor in abacterial prostatitis. British Journal of Urology 54, 729-731 (1982)). FIGS. 5 and 6 respectively show the plasma concentration and cumulative excretion of S-methylcysteine sulfoxide in urine following consumption of Beneforté™ broccoli (described, for example, in WO99/052345) and Stilton soup containing 1.2 mmoles of S-methylcysteine sulfoxide. The study results showed that up to 163.9 μM S-methylcysteine sulfoxide is excreted within the first 0-2 hours.

The present inventors showed that, following consumption of Beneforté™ broccoli and Stilton soup containing 1.0-1.5 mmoles of S-methylcysteine sulfoxide for 12 months, sulfate accumulates in non-tumour prostate tissue of patients whilst the level of adenosine diphosphate (ADP) increases (FIGS. 3 and 4). Another study in prostate cancer patients revealed that the change in cancer-positive cores was highly correlated with the change in ADP and sulfate, such that an increase in ADP correlated with a decrease in cancer-positive cores (FIG. 8). Based on these results, the inventors of the present invention hypothesised that the increase in the levels of ADP were likely to reflect a decrease in the levels of ATP, and that this was linked to the consumption of ATP during the degradation of S-methylcysteine sulfoxide to sulfate derivatives and/or sulfate. This hypothesis was then confirmed with in vitro studies showing that the levels of ATP in non-cancer prostate cells (PNT1A cells) were reduced following 24 hours exposure to a concentration of S-methylcysteine sulfoxide of 100 μM to 200 μM (FIG. 7).

Adenosine triphosphate (ATP) is the main source of energy in both normal and cancer cells, and is required for cell proliferation. In contrast to normal differentiated cells, which rely primarily on mitochondrial oxidative phosphorylation to generate the energy needed for cellular processes, most cancer cells instead rely on aerobic glycolysis, a phenomenon termed “the Warburg effect” to generate ATP (Matthew G. Vander Heiden et al. “Understanding the Warburg Effect: The Metabolic Requirements of Cell Proliferation” Science 324(5930), 1029-1033 (2009)).

Previous studies indicated that cancer cell death can be induced by ATP depletion, although ATP depletion appears to be a means to stop proliferation rather than for tumour regression (Martin D. S. et al. ATP Depletion +Pyrimidine Depletion Can Markedly Enhance Cancer Therapy: Fresh Insight for a New Approach. Cancer Res 60, 6776-6783 (2000)). An ATP-depleting agent in combination with an anti-cancer therapy such as chemotherapy or radiotherapy would likely convert a palliative treatment into a curative treatment (Martin D. S. et al. Cancer Res 60, 6776-6783 (2000)). It was found that ATP depletion activates cell death by necrosis, as opposed to the apoptotic pathway resulting from chemotherapy and radiotherapy. However, cell death by apoptosis requires ATP whilst ATP depletion leads to necrosis, hence these two cell death pathways appear opposed in their mechanisms. Nevertheless, researchers showed that both pathways can occur simultaneously in different cells in the same tumour, and therefore, apoptosis and necrosis can function cooperatively (Martin D. S. et al. Cancer Res 60, 6776-6783 (2000)). Accordingly, it is believed that ATP depletion would enhance cell death by necrosis and improve therapeutic results when used in combination with chemotherapy or radiotherapy.

Another study stated that when ATP is depleted, healthy cells stop proliferating and restock ATP before entering into proliferation again. However, it was observed that cancer cells with active Myc oncoprotein are not able to stop cell proliferation and ATP depletion leads to activation of AMP kinase (AMPK) and p53. This triggers a cascade of events leading to cell death by apoptosis (University of Helsinki “Cancer cell metabolism kills: Possible new therapies targeting energy supply of cancer cells?.” ScienceDaily. 15 Apr. 2013).

In another study, cell death was due to autophagy, a process which is used by the cells as a protective pathway when, for example, levels of ATP decrease. In this study it was revealed that depletion of ATP in prostate cancer cells by inhibition of the aerobic glycolytic metabolism induced cell death by autophagy; and when the aerobic glycolytic metabolism inhibitor was used in combination with an hepatic glucose inhibitor, the cell death pathway shifted from autophagy to apoptosis (Sahra I. B. et al. Targeting Cancer Cell Metabolism: The Combination of Metformin and 2-Deoxyglucose Induces p53-Dependent Apoptosis in Prostate Cancer Cells. Cancer Res 70(6), 2485-2475 (2010)). Autophagy was also observed in prostate cancer cells treated with Salinomycin due to cellular ATP level decrease (Jangamreddy J. R. et al. Salinomycin induces activation of autophagy, mitophagy and affects mitochondrial polarity: Differences between primary and cancer cells. Biochimica et Biophysica Acta 1833, 2057-2069 (2013)).

In vivo, the depletion of ATP occurs throughout the prostate tissue, in both “healthy” and cancer tissues. However, cancer cells are much more sensitive to ATP depletion than normal cells which can regulate their metabolism. Thus, ATP depletion selectively kills cancer cells, and it is the ATP depletion in the tumour micro-environment which is considered to be critical for the treatment of prostate cancer.

Without wishing to be bound by any theory, the inventors consider that the metabolism of S-methylcysteine sulfoxide in prostate tissues is therefore capable of inducing prostate cancer cell death by depleting ATP levels. Hence, S-methylcysteine sulfoxide is suitable to be used for the treatment or prevention of prostate cancer.

Accordingly, in a first aspect, the invention provides a composition comprising S-methylcysteine sulfoxide, an analogue, derivative or metabolite thereof, or a pharmaceutically acceptable salt thereof, for use in the treatment and/or prevention of prostate cancer. Preferably, said composition is for use in the treatment and/or prevention of early-stage prostate cancer. In one embodiment, suitable “early-stage prostate cancer” candidate patients for treatment and/or prophylactic or preventative treatment are those who have been referred for a test to collect a sample of tissue e.g. by a transperineal template biopsy, when there are concerns about possible prostate cancer. In another embodiment of any aspect of the invention, the composition is for administering to men having a diagnosis of low and intermediate localised prostate cancer to prevent further development of aggressive disease. For prevention, the composition in accordance with the invention may also be consumed prophylactically by men who currently lack a cancer diagnosis. Advantageously, the present invention provides a composition that may either eliminate cancerous clones or prevent them from proliferating, thus allowing cancer progression to be intercepted while it is still localised therefore aiming to prevent aggressive disease and metastasis. The term “analogue or derivative thereof” refers to compounds analogous to S-methylcysteine sulfoxide (methiin) or compounds derived from S-methylcysteine sulfoxide, including, for example, selenium analogues such as Se-methylselenocysteine (methylseleniumcysteine) (MSC), and metabolites of S-methylcysteine sulfoxide. Other S-alk(en)yl-L-cysteine sulfoxides include S-propyl-L-cysteine sulfoxide (poplin), trans-S-1-propenyl-L-cysteine sulfoxide(isoalliin), S-(2-propenyl)-L-cysteine sulfoxide (alliin), S-ethyl-L-cysteinesulfoxide (ethiin), S-butyl-L-cysteine sulfoxide (butiin), S-(3-pentenyl)-L-cysteine sulfoxide, S-(1-butenyl)-L-cysteine sulfoxide (homoisoalliin), S-(methylthiomethyl)-L-cysteine sulfoxide (marasmin) and S-(2-pyrrolyl)-L-cysteine sulfoxide. Suitably, such compounds may be derived from Allium species. S-methylcysteine sulfoxide, or any of its analogues or derivatives may be converted into one or more derivatives or metabolites thereof in the body of the subject. In some embodiments, it may be the derivatives or metabolites of S-methylcysteine sulfoxide or its analogues or derivatives that achieve the advantageous effects of the present invention. Metabolites of S-methylcysteine sulfoxide are described, for example in FIG. 1 and also in Edmands et al. (Identification of urinary biomarkers of cruciferous vegetables consumption by metabolic profiling Journal of Proteomic Research 10, 4513-4521 (2011)), and Waring et al.(2003, as above)). Accordingly in another embodiment, the invention provides a metabolite such as those described in FIG. 1 for use in the prevention or treatment of prostate cancer.

Suitably the S-methylcysteine sulfoxide or any of its analogues or derivatives may be provided in a purified form. Purified forms may be purified from plants of the Brassicales or Asparagales orders, including Brassica (i.e. crucifer) or Allium plants for example.

Alternatively the purified form may be chemically synthesised. S-methylcysteine sulfoxide occurs as diastereomers; the positive (+) isomer (S-methyl-L-cysteine sulfoxide) is the predominantly naturally occurring form present in Brassica and Allium species, the negative (−) isomer is produced in addition to the positive (+) isomer when S-methylcysteine sulfoxide is chemically synthesised. In accordance with any aspect of the invention, the S-methylcysteine sulfoxide may be present as the positive isomer, the negative isomer, or a mixture of both positive and negative isomers.

The invention also provides a combination product comprising S-methylcysteine sulfoxide, an analogue, derivative or metabolite thereof and another component from a high glucosinolate cruciferous vegetable. Suitable high glucosinolate cruciferous vegetables are described, for example in WO99/052345. Accordingly, in another aspect, the invention provides, a combination of a) a composition comprising glucoraphanin (4-methylsulphinylbutyl glucosinolate), an analogue, derivative or metabolite thereof; and b) S-methylcysteine sulfoxide, an analogue, derivative or metabolite thereof for use in the treatment and/or prevention of prostate cancer. Without wishing to be bound by any theory, it is suggested that the combined administration of S-methylcysteine sulfoxide and glucoraphanin may provide an enhanced, synergistic or additive effect, with glucoraphanin modulating metabolic processes in the liver that have a systemic effect to enhance metabolic health and S-methylcysteine sulfoxide having an effect on the prostate gland. In one embodiment, a glucoraphanin metabolite is a sulforaphane compound such as isothiocyanate sulforaphane.

In another embodiment, the invention provides a combination product comprising 5-methylcysteine sulfoxide, an analogue, derivative or metabolite thereof and a component from an allium species. In another embodiment, a combination product comprising S-methylcysteine sulfoxide, an analogue, derivative or metabolite thereof may further comprise a compound derived from a tomato.

In one embodiment of any aspect of the invention, there is provided a pharmaceutical composition comprising a composition or combination in accordance with the invention.

Suitably, said pharmaceutical composition comprises one or more pharmaceutically acceptable carriers, diluents or excipients. Acceptable carriers or diluents for use in pharmaceutical formulations are well known in the art and are described, for example, in Remington's Pharmaceutical Sciences, Mack Publishing Co. (A. R. Gennaro edit. 1985).

In another embodiment a composition comprising S-methylcysteine sulfoxide or its analogue, derivative or metabolite thereof, or a combination product in accordance with any aspect of the invention may be administered as a food composition. A food composition includes but is not limited to a soup, a juice, a smoothie, a spread, a yogurt, a sauce, a gravy, a tart, a quiche, a pie, a snack food bar, a prepared vegetable product and a blended product. In one embodiment, the composition or combination in accordance with any aspect of the invention, when used as or in the preparation of food, may be used in conjunction with one or more of: a nutritionally acceptable carrier, a nutritionally acceptable diluent, a nutritionally acceptable excipient, a nutritionally acceptable adjuvant, a nutritionally active ingredient.

The food composition may be fresh, or it may be treated to extend its shelf life. Methods of extension of shelf life of the food composition may include drying, chilling, freezing, the addition of suitable food grade preservatives, pasteurisation or otherwise sterilisation.

In one embodiment, a food composition in accordance with the invention is or comprises a portion of an edible SMCSO-containing plant, for example a Brassica, crucifer or allium vegetable.

In one embodiment a food composition in accordance with the invention is broccoli soup. In another embodiment, the food composition may further comprise additional food components such as a food from an allium species, a tomato species and so forth. The inclusion of additional food components with a low pH, for example additional food components from a tomato species, is advantageous because this leads to a reduction in the pH of the food composition, aiding the optional process of pasteurisation and extending the shelf life of the food composition.

In one embodiment the food composition comprises S-methylcysteine sulfoxide or its analogue, derivative or metabolite thereof. Suitably the S-methylcysteine sulfoxide or its analogue, derivative or metabolite thereof is provided in a purified form. S-methylcysteine sulfoxide may be analysed using for example the method of Bernaert et al. (Bernaert, N., et al., Influence of cultivar and harvest time on the amounts of isoalliin and methiin in leek (Allium ampeloprasum var. porrum). J Agric Food Chem, 2012. 60(44): p. 10910-9).

In another embodiment, the food composition is a functional food composition or nutraceutical composition. As used herein, the term “functional food” or “nutraceutical” means food which is capable of providing not only a nutritional effect and/or a taste satisfaction, but is also capable of delivering a further beneficial effect to the consumer, for example specific health effects.

In some embodiments, the food composition may be a drink or a beverage. In another embodiment, said drink or beverage is obtained from a reconstituted powdered composition. Said powder may be packaged in a sachet and reconstituted as a drink or beverage by a subject when required or desired. In a further embodiment, said drink or beverage is obtained from a reconstituted effervescent tablet composition. Said effervescent tablet may be reconstituted as a drink or beverage by a subject when required or desired.

In another embodiment, the food composition is a food ingredient. As used herein the term “food ingredient” includes a formulation which is or can be added to a food composition such as, for example, functional foods or nutraceuticals or foodstuffs as a nutritional supplement and/or fibre supplement. Said functional foods or nutraceuticals or foodstuffs may be used as staple foods as well as under clinical regimen.

In another embodiment, the food composition is a food supplement.

Suitably, the food composition in accordance with the invention is for use in the treatment and/or prevention of prostate cancer, preferably, early-stage prostate cancer. In another embodiment, said food composition comprises one or more suitable carriers.

In one embodiment of any aspect of the invention, the S-methylcysteine sulfoxide or its analogue, derivative or metabolite thereof, may be added to a food composition as an additive.

The routes of administration of a pharmaceutical and/or food composition in accordance with the invention include, but are not limited to, the following: oral, topical, mucosal, nasal, parenteral, gastrointestinal, intradermal, intracranial, intratracheal, subcutaneous, transdermal, rectal, buccal, sublingual. Preferably, compositions or combinations including pharmaceutical and/or food compositions in accordance with the invention are administered orally in the form of tablets, powder, capsules, ovules, elixirs, solutions or suspensions. The formulation used in the pharmaceutical and/or food compositions may control the release of the active compounds. For example, immediate-, delayed-, modified-, sustained-, pulsed- or controlled-release. Said control-release formulation may comprise auxiliary additives such as, for example, a carrier, diluent or solubiliser. Said control-release formulation may also comprise complexation compounds such as, for example, cyclodextrins. Drug-cyclodextrin complexes may be suitable for most dosage forms and administration routes. Suitable control-release formulations may enhance delivery to the prostate gland.

Compositions according to the invention may be formulated as powders, granules or semisolids for incorporation in capsules. For semisolid formulations, active compounds may be diluted in a semisolid carrier such as, for example, polyethylene glycol or a liquid carrier such as, for example, glycol. Powders and/or granules may be obtained by freeze-drying and/or spray-drying of, for example, a cruciferous and/or allium vegetable extract. Solid compositions such as tablets may contain excipients such as micro-crystalline cellulose (MCC), dextran, lactose, sodium citrate, calcium carbonate, dibasic calcium phosphate and glycine, disintegrants such as starch, sodium starch glycollate, croscarmellose sodium, silicates, granulation binders such as polyvinylpyrrolidone, hydroxypropylmethyl cellulose (HPMC), hydroxypropylcellulo se (HPC), sucrose, gelatin and acacia, and lubricating agents such as magnesium stearate, stearic acid, glyceryl behenate and talc. Similar solid compositions may be used as fillers for gelatin capsules.

Preferably a composition or combination product in accordance with the invention may have an extended shelf life at ambient temperature.

The skilled person will be familiar with methods for determining a suitable effective dose of a composition or combination in accordance with the invention. An effective dose is generally one which induces the desired physiological effect. In the context of the prevention and/or treatment of prostate cancer, a desired physiological effect is the reduction in the overall rate of progression of prostate cancer. In a prophylactic or preventative treatment, this may be considered to be an absence of progression to prostate cancer. In a treatment of early prostate cancer, this may be considered to be a reduction in the rate of progression of prostate cancer, e.g. in a population study, this may be a reduced rate of progression of prostate cancer in a treated population compared to non-treated population. In one embodiment, reduced progression or a reduced rate of progression may be measured by taking needle core biopsies and analysing using histopathology to determine the presence or absence of prostate cancer cells. In another embodiment, reduced progression or a reduced rate of progression may be measured by attributing a Gleason score or classification to the cancer cells isolated by prostate biopsy. In another embodiment, a reduced rate of progression may be determined by observing a decrease in the number of individuals needing other interventions for prostate cancer (e.g. surgery and/or chemotherapy) in a treated population compared to an untreated population. In one embodiment, the treated population is a population of men who are under “active surveillance” for a prostate condition. In such an untreated population under “active surveillance”, the proportion of men needing follow on surgery or drug intervention is generally in the region of approximately 30% per year. Accordingly, in a treated population, the proportion may be expected to be less than 30% per year. In another embodiment the desired physiological effect may be a reduced number of cancer foci in the prostate gland.

In one embodiment, an effective dose for a composition or combination in accordance with the invention provides a dose of active component in the region of 0.2 to 3 mmmoles. Accordingly, in one embodiment, a pharmaceutical composition in accordance with the invention provides a dose in the region of about 0.20 to about 3 mmoles of S-methylcysteine sulphoxide per dose. In another embodiment, said pharmaceutical composition comprises 1.0-1.5 mmoles of S-methylcysteine sulfoxide per dose. In another embodiment a pharmaceutical composition provides a dose in the region of about 0.2 to about 3 mmmoles of MSC per dose.

In another embodiment, an effective dose for a composition or combination in accordance with any aspect of the invention provides a dose of active component in the region of 3 to 50 mmoles, suitably at least 3 mmoles, preferably more than 10 mmoles, more than 20 mmoles, more than 30 mmoles, more than 40 mmoles or more than 50 mmoles. Accordingly, in one embodiment, a pharmaceutical composition in accordance with the invention provides a dose in the region of about 3 to about 50 mmoles of S-methylcysteine sulphoxide per dose, preferably about 3 to about 25 mmoles S-methylcysteine sulphoxide per dose. . In another embodiment a pharmaceutical composition provides a dose in the region of about 3 to about 50 mmmoles of MSC per dose.

In another embodiment, a single dose of a food composition in accordance with the invention comprises about 0.20 to about 3 mmoles of S-methylcysteine sulfoxide. In another embodiment said food composition comprises 1.0-1.5 mmoles of S-methylcysteine sulfoxide. In another embodiment a single dose of a food composition in accordance with the invention comprises about 0.20 to about 3 mmoles of MSC. In another embodiment said food composition comprises 1.0-1.5 mmoles of MSC.

In another embodiment, a single dose of a food composition in accordance with the invention comprises about 3 to about 50 mmoles of S-methylcysteine sulfoxide, preferably about 3 to about 25 mmoles S-methylcysteine sulphoxide. In another embodiment, a food composition in accordance with the invention provides a dose of at least 3 mmoles, preferably more than 10 mmoles, more than 20 mmoles, more than 30 mmoles, more than 40 mmoles or more than 50 mmoles of S-methylcysteine sulphoxide. In another embodiment a single dose of a food composition in accordance with the invention comprises about 3 to about 50 mmoles of MSC.

A suitable dose for a composition or combination in accordance with the invention may be found by reference to the Examples herein.

The amount of S-methylcysteine sulfoxide or a composition thereof to be administered to a subject will depend on the biological activity and bioavailability which will vary depending on the formulation, the mode of administration, as well as whether it is administered as a monotherapy or in a combined therapy. The frequency of administration will also be dependent on the aforementioned factors as well as on the half-life of S-methylcysteine sulfoxide within the subject being treated. In an embodiment, the subject is administered with S-methylcysteine sulfoxide or a composition thereof once per week such that said subject is administered a total weekly dose of S-methylcysteine sulfoxide of about 0.20 to 3 mmoles, preferably 1.0-1.5 mmoles. In another embodiment, the subject is administered with S-methylcysteine sulfoxide or a composition thereof once per week such that said subject is administered a total weekly dose of S-methylcysteine sulfoxide of at least 3 mmoles, preferably more than 10 mmoles, more than 20 mmoles, more than 30 mmoles, more than 40 mmoles or more than 50 mmoles. It will be appreciated that a single weekly dose may be replaced by any number of individual daily etc. doses to give the same concentration effect. For example, the S-methylcysteine sulfoxide may be administered as a single dose per week, or as multiple doses per week, for example two, three, four, five or more doses per week. In the case of a combined therapy, S-methylcysteine sulfoxide or a composition thereof and the other anti-cancer therapy with which it is combined may be administered by different routes and at different dosages and frequencies. Known procedures such as in vivo experimentation and clinical trials may be used to establish specific therapeutic regimes, such as dosage and frequency.

The pharmaceutical and food compositions of the invention may further comprise all desired components and/or additives which are suited for use in pharmaceuticals or food including flavourings, colourings, preservatives, sugar, minerals, vitamins, fibres, buffering agents (e.g. citrate or phosphate buffers), effervescent agents (this includes an acidic component such as, for example, citric or tartaric acid and an alkaline component such as, for example, sodium carbonate, potassium carbonate or calcium carbonate, or sodium bicarbonate and potassium bicarbonate, in whose reaction with one another gaseous carbon dioxide is formed; an alternative to the acids mentioned is the use of the acidic salts or mixtures of the acids with the salts), antioxidants, nutritional compounds, etc., as long as they do not affect the stability of S-methylcysteine sulfoxide present therein.

Known procedures such as in vivo experimentation and clinical trials may be used to establish specific formulations of pharmaceutical and/or food compositions.

In one embodiment of any aspect or embodiment of the invention, S-methylcysteine sulfoxide, or its analogues or derivatives, may be obtained from any suitable source. S-methylcysteine sulfoxide, or its analogues or derivatives, may be obtained or purified from plants of the Brassicales or Asparagales orders, including Brassica or Allium plants for example. S-methylcysteine sulfoxide or its analogues or derivatives may be derived from cruciferous and/or allium vegetables, portions thereof, extracts thereof or combinations thereof. Preferably, S-methylcysteine sulfoxide or its analogues or derivatives is derived from brassica vegetables, portions thereof, extracts thereof or combinations thereof. More preferably, S-methylcysteine sulfoxide or its analogues or derivatives is derived from a broccoli plant, portions thereof, extracts thereof or combinations thereof. Vegetables rich in particular compounds may be generated through genetics and agronomy including breeding programs. For MSC, vegetables such as broccoli may be fertilised with selenium as brassica vegetables accumulate selenium. In particular, broccoli (e.g. heterozygous for Myb28^{villosa}) is particularly effective at accumulating selenium as MSC without any effect on glucosinolate accumulation (Doberstein, The Effects of sulphur and selenium on glucoraphanin and seleno-methylselenocysteine concentration in broccoli. 2015, University of Minnesota).

Methods and compositions relating to the bioactive components of brassica vegetables as well as methods for modification the composition of brassica to increase content of different bioactive compounds, and their use are described, for example, in WO2010/001119, WO2011/077163, WO99/27120, WO 99/52345, WO 2011/077163, US2014/0075590 and US2014/0189905.

In another embodiment, S-methylcysteine sulfoxide or its analogues or derivatives may be substantially isolated and/or purified.

Likewise, glucoraphanin, its analogues or derivatives for use in a combination product in accordance with any aspect or embodiment of the invention may also be derived from any suitable source including cruciferous and/or allium vegetables, portions thereof, extracts thereof or combinations thereof. Purified forms may be obtained or purified from plants of the Brassicales or Asparagales orders, including Brassica or Allium plants for example Preferably, glucoraphanin or its analogues or derivatives is derived from brassica vegetables, portion thereof, extract thereof or combinations thereof. More preferably, glucoraphanin or its analogues or derivatives is derived from a broccoli plant, portion thereof, extract thereof or combinations thereof. In another embodiment, glucoraphanin or its analogues or derivatives may be substantially isolated and/or purified.

Cruciferous vegetables containing S-methylcysteine sulfoxide or its analogues or derivatives and/or glucoraphanin include, but are not limited to, the following cruciferous vegetable crops:

Broccoli,

Cabbage,

Collards,

Kale,

Brussels sprouts,

Cauliflower,

Turnip,

Swede,

Mustard seeds

Kalettes™

Broccoli also contains multiple health-promoting compounds, including vitamins A, C and K, flavonoids, selenium and secondary metabolites like glucosinolates (Moreno D. A. et al. Chemical and biological characterisation of nutraceutical compounds of broccoli. Journal of Pharmaceutical and Biomedical Analysis 41, 1508-1522 (2006)).

Allium vegetable crops containing S-methylcysteine sulfoxide and its analogues or derivatives include, but are not limited to, the following:

Onion,

Leek,

Garlic,

Shallots,

Chives.

The term “cruciferous and/or allium vegetable(s)” as used herein may refer to fresh cruciferous and/or allium vegetable(s), processed cruciferous and/or allium vegetable(s), portions thereof, extracts thereof or combinations thereof.

The term “fresh cruciferous and/or allium vegetable(s)” as used herein refers to cruciferous and/or allium vegetable(s) consumed raw or cooked. It is known that S-methylcysteine sulfoxide is thermally degraded upon cooking to produce several volatile S-containing compounds which are the major contributor to the flavour of cooked brassica vegetables (Traka M. H. et al. Genetic regulation of glucoraphanin accumulation in Beneforté® broccoli. New Physiologist (2013)). Accordingly, preferably, fresh cruciferous and/or allium vegetable(s) are consumed raw.

The term “processed cruciferous and/or allium vegetable(s)” as used herein refers to cruciferous and/or allium vegetable(s) having been subject to at least one further processing step such as maceration, drying, freezing, compacting, etc.

The term “extract thereof” as used herein refers to the substance or mixture of substances obtained from extraction of the cruciferous and/or allium vegetable(s). The extraction may be carried out by mechanical or chemical action. Examples of extraction methods include the use of pressure distillation, evaporation, dissolution in solvents. The extract may be a crude extract. In one embodiment, the extract comprises at least S-methylcysteine sulfoxide from the cruciferous and/or allium vegetable(s). In a preferred embodiment, the extract is an alcoholic (e.g. ethanol) or aqueous extract. More preferably, the extract is an aqueous extract.

The term “portion thereof” as used herein refers to any part of the cruciferous and/or allium vegetable(s). A portion thereof may refer to but is not limited to florets, inflorescences, seeds, leaves, bulbs, roots and/or stems.

In some embodiments, S-methylcysteine sulfoxide and pharmaceutical and food compositions thereof of the present invention may be supplemented with natural and/or synthetic S-methylcysteine sulfoxide.

S-methylcysteine sulfoxide or its analogues or derivatives may be of a natural or synthetic origin. In an embodiment, S-methylcysteine is from synthetic origin. S-methylcysteine sulfoxide (CAS number 6853-87-8) is commercially available from, for example, LKT Laboratories, Inc.

The term “substantially isolated” used herein means that a compound is free from other components such as contaminants. Substantially isolated means that the isolate comprises at least 80% concentration w/w of a compound. Preferably, the isolate comprises at least 90% concentration w/w of a compound. More preferably, the isolate comprises at least 95% concentration w/w of a compound. More preferably, the isolate comprises at least 99% concentration w/w of a compound.

The term “substantially purified” used herein means that a compound has undergone a purification process whereby other components, such as contaminants, are removed. The term “substantially purified” means that the purified product comprises at least 90% concentration w/w of a compound. Preferably, the purified product comprises at least 95% concentration w/w of a compound. More preferably, the purified product comprises at least 99% concentration w/w of a compound.

In another embodiment, there is provided a composition or combination in accordance with the invention which can also be used in combination with another anti-cancer therapy such as chemotherapy, hormone therapy or radiotherapy. Such combination can enhance the effects of chemotherapy, hormone therapy or radiotherapy. Accordingly, in another embodiment, a composition or combination in accordance with the invention is used in combination with another anti-cancer therapy for the treatment of prostate cancer. In another embodiment, said anti-cancer therapy is chemotherapy, hormone therapy or radiotherapy. Prostate cancer chemotherapy agents includes, but are not limited to, docetaxel (Taxotere®), cabazitaxel (Jevtana®), mitoxantrone (Novantrone®), estramustine (Emcyt®), doxorubicin (Adriamycin®), etoposide (VP-16), vinblastine (Velban®), paclitaxel (Taxol®), carboplatin (Paraplatin®) and vinorelbine (Navelbine®). Hormone therapy agents include, but are not limited to, luteinizing hormone-releasing hormone (LHRH) analogues (e.g. degarelix (Firmagon®), abiraterone (Zytiga®)) and anti-androgens (e.g. enzalutamide (Xtandi®)). In one embodiment, a composition or combination in accordance with the invention and said other anti-cancer therapy may be administered by different routes and at different dosages and frequencies.

The term “subject” used herein refers to vertebrates, particularly to mammalians. The term includes but is not limited to domestic animals, sports animals, primates and humans. Preferably, the subject is a human. The term “patient” used herein refers to humans.

In another aspect, the invention provides for the use of a composition comprising S-methylcysteine sulfoxide, an analogue, derivative or metabolite thereof, or a combination of a) a composition comprising glucoraphanin, an analogue, derivative or metabolite thereof; and b) S-methylcysteine sulfoxide, an analogue, derivative or metabolite thereof, in accordance with any embodiment of the invention, for the manufacture of a medicament for use in the treatment and/or prevention of prostate cancer. Preferably, said prostate cancer is early-stage prostate cancer.

In another aspect, the invention provides a method of treatment and/or prevention of prostate cancer comprising administering a therapeutically effective amount of a composition comprising S-methylcysteine sulfoxide, an analogue, derivative or metabolite thereof, or a combination of a) a composition comprising glucoraphanin, an analogue, derivative or metabolite thereof; and b) S-methylcysteine sulfoxide, an analogue, derivative or metabolite thereof, in accordance with any embodiment of the invention, to an individual in need thereof. Preferably, said prostate cancer is early-stage prostate cancer.

Suitably, said composition or combination is a pharmaceutical composition. In another embodiment, said pharmaceutical composition or combination comprises one or more pharmaceutically acceptable carriers, diluents or excipients.

In another aspect, there is provided a method of treatment of prostate cancer in a subject in need thereof comprising administering to said subject a therapeutically effective amount of a) a composition comprising glucoraphanin, an analogue, derivative or metabolite thereof; and b) S-methylcysteine sulfoxide, an analogue, derivative or metabolite thereof.

In one embodiment, a combination product or pharmaceutical composition comprising a combination of a) and b) in accordance with any embodiment of the invention can be provided for administration sequentially or simultaneously in separate or combined compositions.

In another embodiment, the invention provides for the use of a food composition comprising S-methylcysteine sulfoxide for the manufacture of a food composition for the treatment and/or prevention of prostate cancer. Preferably, said prostate cancer is early-stage prostate cancer. In another embodiment, said food composition comprises one or more suitable carrier(s).

In a further embodiment, the invention provides for a method of treatment and/or prevention of prostate cancer comprising administering a therapeutically effective amount of a food composition comprising S-methylcysteine sulfoxide. Preferably, said prostate cancer is early-stage prostate cancer. In another embodiment, said food composition comprises one or more suitable carrier.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the following Figures and Examples.

### FIGURES

FIG. 1. Human Metabolism of S-methylcysteine sulfoxide

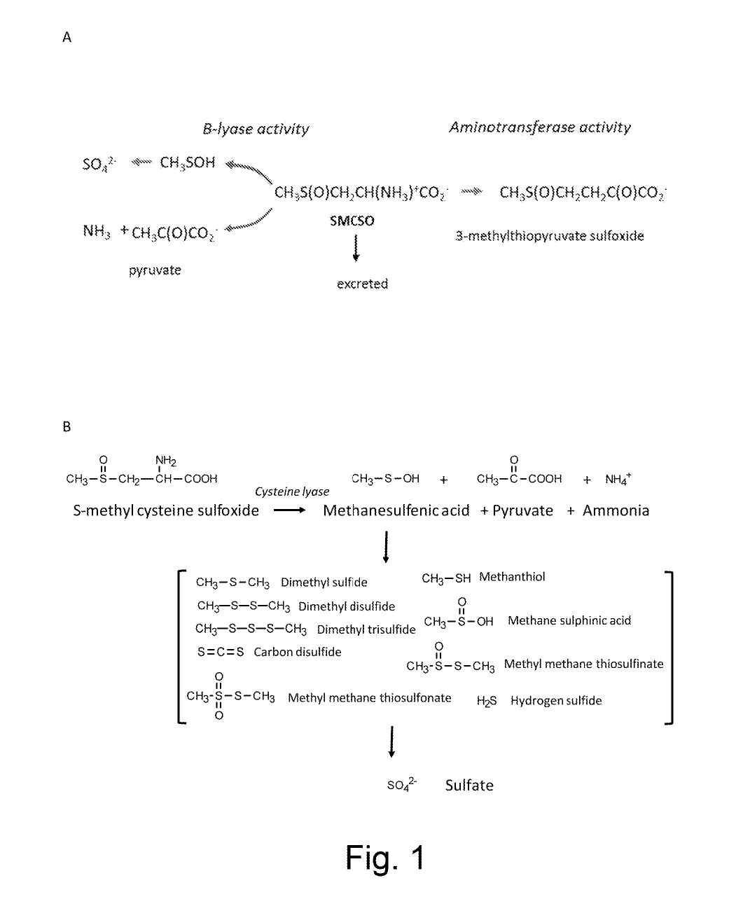

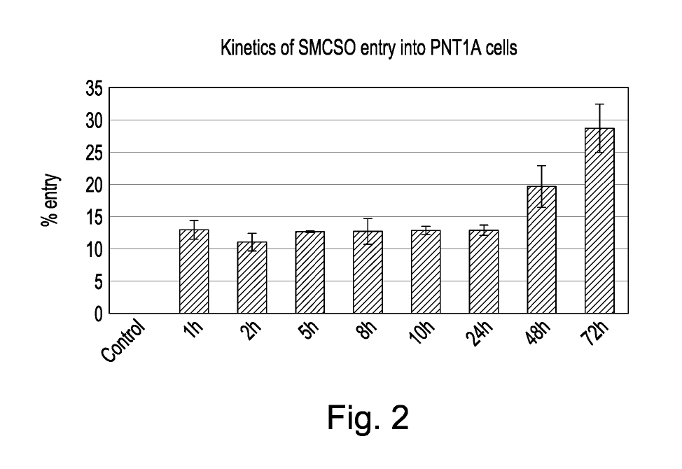

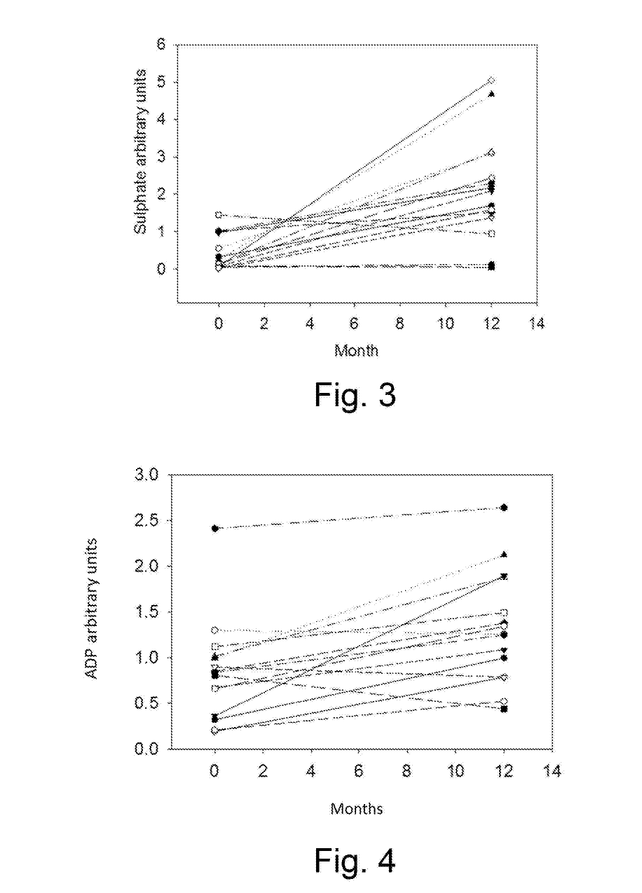

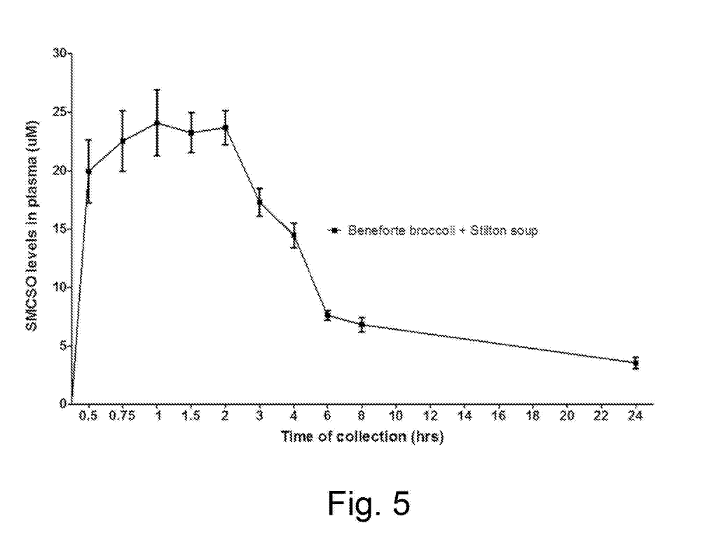

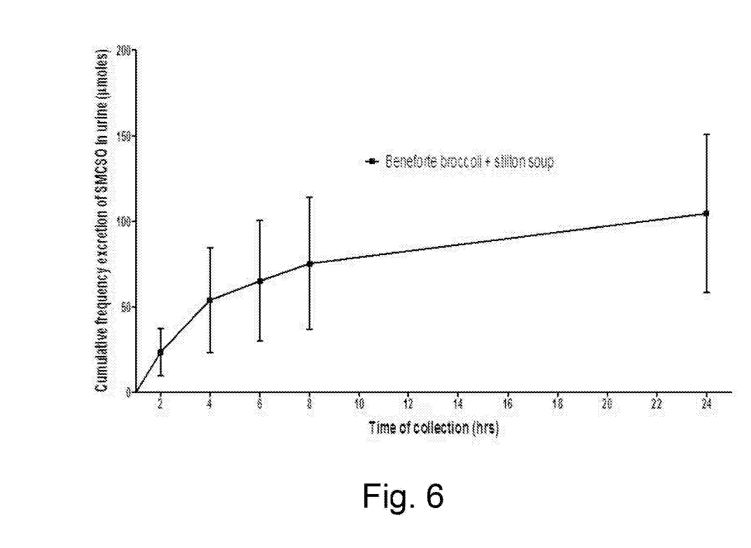

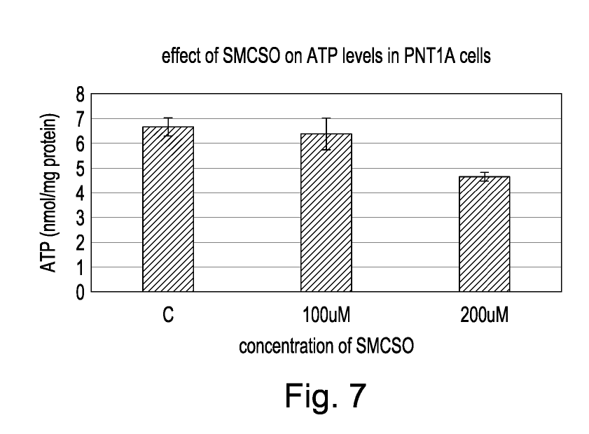

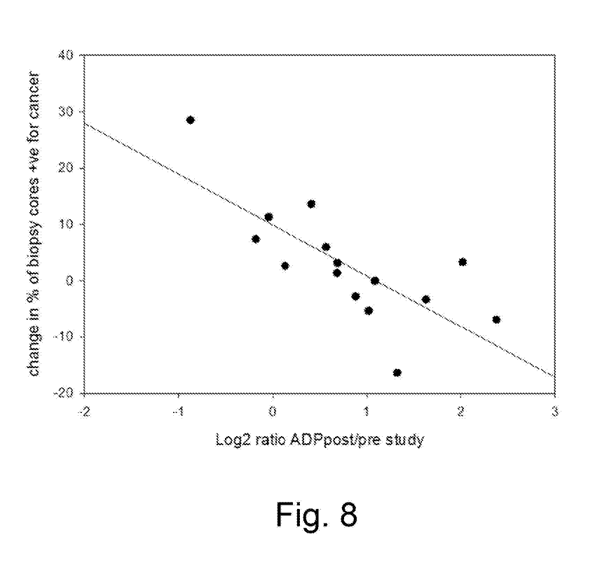

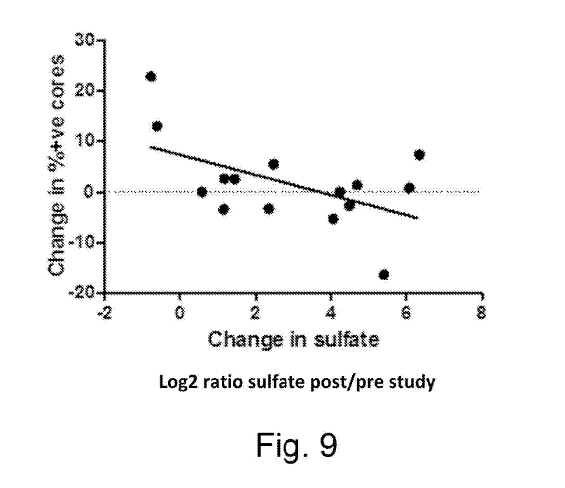

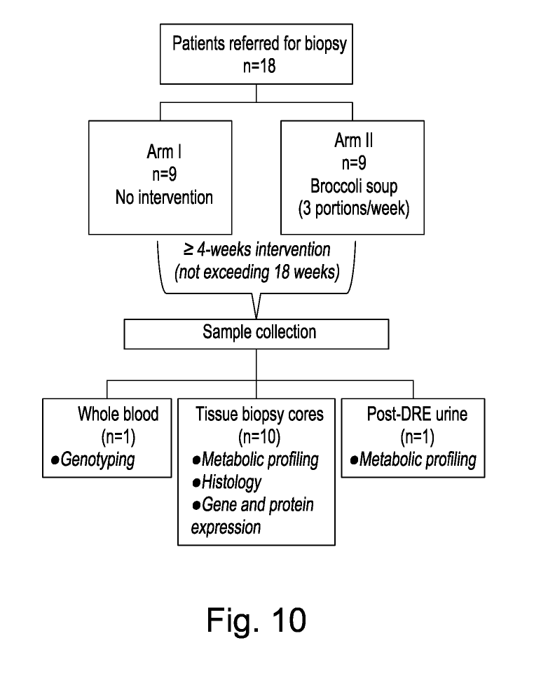

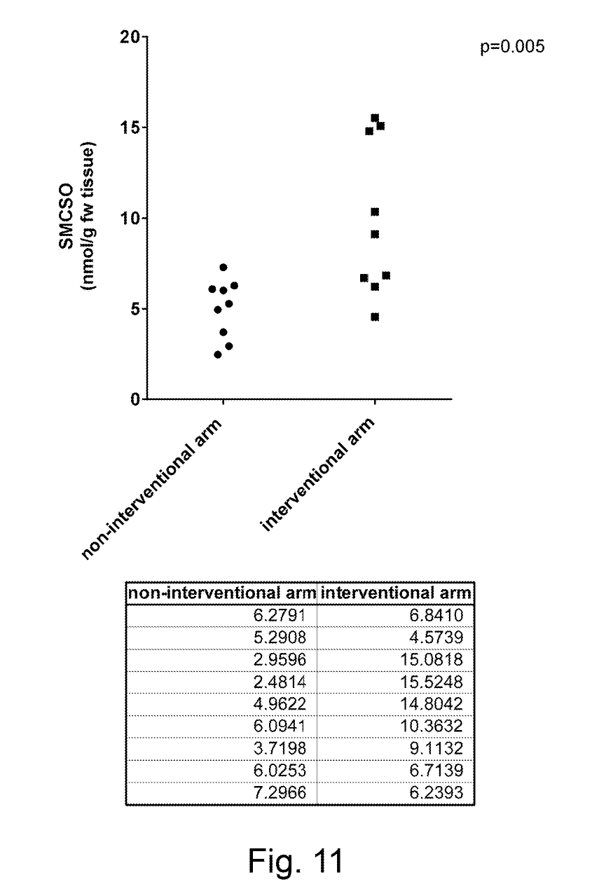

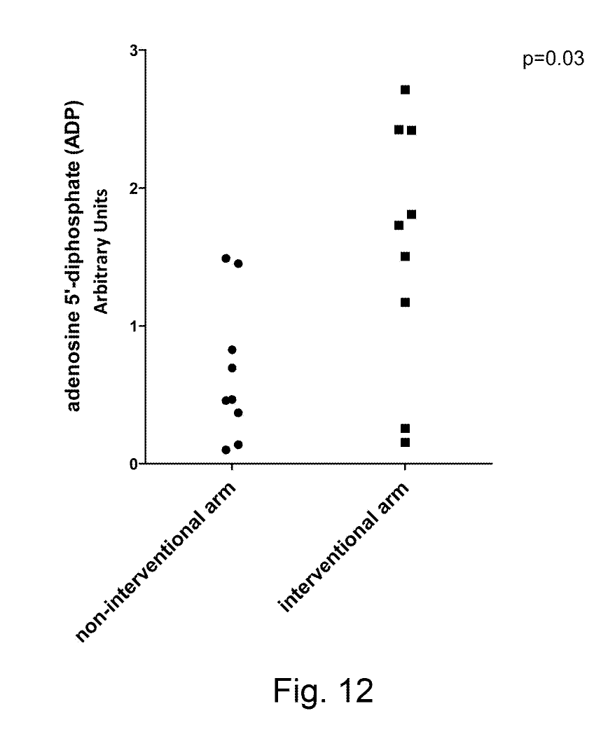

The flowchart in FIG. 1A represents the pathways of S-methylcysteine sulfoxide following administration of radioactive S-methylcysteine sulfoxide to humans. Following consumption of SMCSO, 6-8% of SMCSO was excreted un-metabolised within urine within 24 h, and it is likely that additional SMCSO is excreted following N-acetylation. Within tissue, two important metabolic routes is firstly beta-lyase activity which will result in sulfate and pyruvate, and secondly aminotransferase activity which will result in an alpha-keto acid.

The flowchart in FIG. 1B shows how the action of cysteine lyase on S-methylcysteine sulfoxide results in formation of short lived methanesulfenic acid that is metabolised to a variety of derivatives, some of which shown in the figure, and some of which are oxidised to sulfate.

FIG. 2. Rapid SMCSO Entry into Human Prostate Cells.

PNT1A cells were treated with 300μM SMCSO and intracellular levels of SMCSO were quantified following incubation of between 1 h and 72 h. Data shown are means±SD (n=3).

FIG. 3. Accumulation of Sulfate in Non-Tumour Prostate Tissue

This graph shows the accumulation of sulfate in non-tumour prostate tissue following consumption of Beneforté™ broccoli and Stilton soup containing 1.0-1.5 mmoles of S-methylcysteine sulfoxide per week for 12 months. Fifteen patients took part in this study. The level of sulfate in prostate tissue of patients enrolled in this study was measured at 0 and 12 months. Sulphate is likely to be derived from metabolism of SMCSO as shown in FIG. 1.

FIG. 4. Accumulation of ADP in Non-Tumour Prostate Tissue

This graph show the accumulation of ADP in non-tumour prostate tissue following consumption of Beneforté™ broccoli and Stilton soup containing 1.0-1.5 mmoles of S-methylcysteine sulfoxide per week for 12 months. Fifteen patients took part in this study. The level of ADP in prostate tissue of patients enrolled in this study was measured at 0 and 12 months.

FIG. 5. Plasma Concentration of S-methylcysteine sulfoxide

This graph shows the change in plasma concentration of S-methylcysteine sulfoxide following consumption of Beneforté™ broccoli and Stilton soup containing 1.2 mmoles of S-methylcysteine sulfoxide per portion. The change in plasma concentration of S-methylcysteine sulfoxide was recorded over 24 hours in ten patients.

FIG. 6. Cumulative Excretion of S-methylcysteine sulfoxide in Urine

This graph shows the cumulative excretion of S-methylcysteine sulfoxide in urine following consumption of Beneforté™ broccoli and Stilton soup containing 1.2 mmoles of S-methylcysteine sulfoxide. Ten patients took part in this study. The total S-methylcysteine sulfoxide excreted represented 23% of that consumed.

FIG. 7. In Vitro Reduction of ATP in Non-Cancer Prostate Cell Line

This graph shows that the levels of ATP in non-cancer prostate cells (PNT1A cells) were reduced following 24 hours exposure to S-methylcysteine sulfoxide. The concentration of S-methylsulfoxide used in this study (100 μmol to 200 μmop was similar to that found in urine following consumption of 1.0-1.5 mmoles of S-methylcysteine sulfoxide.

FIG. 8. Change in Percentage Biopsy Core Relating to the Levels of ADP

This graph shows that the percentage of biopsy cores that are positive for prostate cancer is negatively correlated with change in ADP (p=0.001). Fifteen patients took part in this study. The change in percentage of cancer-positive cores from biopsy samples taken at the 0 and 12-month time-points of this study, as determined by histopathology, was correlated with the change in ADP concentration at the same time-points. The change in cancer-positive cores was highly correlated with the change in ADP, such that an increase in ADP correlated with a decrease in cancer-positive cores.

FIG. 9. Change in Percentage Biopsy Core Relating to the Levels of sulfate

This graph shows that the percentage of biopsy cores that are positive for prostate cancer is negatively correlated with change in sulfate (p=0.001). Fifteen patients took part in this study. The change in percentage of cancer-positive cores from biopsy samples taken at the 0 and 12-month time-points of this study, as determined by histopathology, was correlated with the change in sulfate concentration at the same time-points. The change in cancer-positive cores was highly correlated with the change in sulfate, such that an increase in sulfate correlated with a decrease in cancer-positive cores.

FIG. 10. The Study Outline for the Short (4-6 weeks) Dietary Intervention Study.

FIG. 11. SMCSO Accumulation in Prostate Tissue Following Short Dietary Intervention Study

This graph shows the accumulation of S-methylcysteine sulfoxide in prostate tissue biopsy cores taken from patients in the non-interventional arm and interventional arm of the short dietary intervention study. Nine patients were in each arm of the study. The graph shows that SMCSO accumulation in prostate tissue is positively correlated with consumption of the broccoli and Stilton soup (p=0.005).

FIG. 12. ADP Accumulation in Prostate Tissue Following Short Dietary Intervention Study

This graph shows the accumulation of ADP in prostate tissue biopsy cores taken from patients in the non-interventional arm and interventional arm of the short dietary intervention study. Nine patients were in each arm of the study. The graph shows that ADP accumulation in prostate tissue is positively correlated with consumption of the broccoli and Stilton soup (p=0.03).

### EXAMPLE

### Example 1

Twelve Month Diet Intervention Study

A twelve-month intervention study, where 51 patients on active surveillance with a confirmed diagnosis of low risk or intermediate risk prostate cancer (Gleason score ≤7, PSA ≤20 μg/l, stage T1c-T2a and T2b-T2c), and not taking 5α-reductase inhibitors or testosterone replacement medicines, took a dietary product comprising 300 g per week of broccoli soup as described below, to assess its effect on prostate cancer development. The patients were evenly divided into three study arms, with one-way ANOVA showing no significant difference between study arms for any variable (age (years); BMI (kg/m2); Systolic BP (mm Hg); Diastolic BP (mm Hg); baseline blood glucose concentration (mmol/L); baseline blood lipid profiles for Cholesterol, LDLc, HDLc, Triglyceride (all mmol/L)). Each arm was provided with a different variant of the broccoli soup, containing either ‘standard’ broccoli, Beneforte™ broccoli (heterozygous for myb28^{villosa}) or Benefort™ Extra broccoli (homozygous for Myb28^{volliosa}) described, for example in Traka et al. (2013). The soup was produced using a standard recipe, incorporating 84 g broccoli (raw weight), 40 g semi-skimmed milk, 25 g fresh whipping cream, 20 g onion (raw weight), 20 g new potato (raw weight), 12 g Stilton cheese, 3 g rapeseed oil, 3 g cornflour, 1.6 g salt and 1.4 g black pepper per 300 g final soup. Routine hospital follow-up data of fasting glucose, lipid profile and prostate-specific antigen concentration (PSA) was measured in blood samples taken 3-4 monthly. In addition, baseline and 12-month samples were taken of prostate biopsy tissue, blood samples and urine samples. Tissue taken by transperineal prostate biopsy (TPB) was stored in either RNAlater™ solution, or in MeOH—H₂O solution, or was snap-frozen. Blood samples were stored either as whole blood, blood plasma or serum. The urine samples were collected post-digital rectal examination (DRE) and stored at −80° C.

Liver and kidney function measurements at 0 and 12 months of intervention showed that no toxicity was associated with the intervention. Histopathological assessment of prostate biopsy tissue of the patients sampled at the 0 and 12 month time-points (n=19) demonstrated that 10 patients' biopsies showed a reduction or no change in the percentage of positive cores over time, 10 patients' biopsies showed no change in Gleason score of cancerous tissue.

### Example 2

Levels of SMCSO in Broccoli Soup

Measurements were made of the concentration of SMCSO in broccoli soup prepared using different broccoli starting materials. Total SMCSO fractions were prepared from each sample and measurements of SMCSO concentration were conducted by HPLC essentially according to the method of Traka et al (2013). The concentration of SMCSO in standard broccoli soup was found to be 22.4 μmol per gram dry weight of soup, compared with 32.6 μmol per gram dry weight in Beneforte™ broccoli soup and 36.4 μmol per gram dry weight in Beneforte™ Extra broccoli soup.

### Example 3

Cell Assay Showing that SMCSO Accumulates in Cells.

Human prostate PNT1A cells were incubated with 300 μM SMCSO for up to 72 h in triplicate. Whole cell lysates were collected at regular intervals between 0 and 72 h and levels of SMCSO were quantified by HPLC as above. Cellular uptake of SMCSO is rapid, as early as 1 h, and levels remain constant at 10% of the initial exposure levels for at least 24 h. Uptake increases up to approximately 30% following 72 h of exposure to SMCSO. Results are shown in FIG. 2.

### Example 4

Changes in sulfate and ADP Levels in Prostate Tissue

The level of sulfate in non-tumour prostate tissue of patients enrolled in the 12 month diet intervention study outlined in Example 1 (n=15) was measured at 0 and 12 months of the study as part of a high throughput HPLC-based metabolite profiling undertaken by Metabolon Inc. The consumption of the broccoli soup contributed between 1 and 1.5 mmoles of SMCSO to subjects' diets per week. On average the concentration of sulfate increased, with a number of patients showing an increase in prostatic sulfate concentration (See FIG. 3). This is despite the fact that the additional S that one portion of soup provides per week in the diet is less than 0.2% of total S ingested. In the same samples, ADP concentration was measured (Metabolon Inc.) and showed an increase on average (see FIG. 4). This is assumed to correlate with a concomitant decrease in ATP levels.

### Example 5

SMCSO Bioavailability from Diet Intervention

A dietary intervention study was carried out on healthy subjects (n=10) whereby a single event of dietary supplementation was carried out with Beneforte™ broccoli and stilton soup, produced according to the recipe as described in Example 1, providing an effective SMCSO dose of 1.2 mmoles. This was followed by urine and blood collection over 24 h. Plasma and urinary concentrations of SMCSO were measured, see FIGS. 5 (plasma) and 6 (urine). SMCSO concentration in blood plasma increased from a baseline level of 0 μM at the start of the 24 h period to a maximum concentration of 24 μM one hour after consumption of the soup. Blood plasma levels remained elevated above baseline at the end of the 24 h period. Cumulative urinary SMCSO measurements (FIG. 6) showed that the baseline concentration was 0 μmoles, increasing to a mean urinary concentration of 71.5 μmoles 2-4 hours after dietary supplementation. The 24 h cumulative urinary concentration was over 100 μmoles SMCSO. The concentration of SMCSO in urine between 0-2 h after consumption had a concentration of up to 164 uM, and was likely to >200 uM within a single urination.

### Example 6

SMCSO Leads to Reduction in ATP Levels in Prostate Cell Assay.

Cells of the human prostatic PNT1A cell line were incubated with SMCSO at 100 μM or 200 μM, these concentrations were chosen as they were consistent with the physiological concentrations of SMCSO measured in the human intervention diet study, for example 2-4 hours after consumption of Beneforté™ broccoli soup the concentration of SMCSO in the urine of study subjects ranged from 42.18 μM to 130.17 μM, see Table 1. ATP levels (nmol/mg protein) in control and SMCSO treated cells were measured fluorimetrically using a commercially available kit (Abcam, ab 8335). The cellular concentration of ATP was shown to be reduced in the presence of SMCSO (see FIG. 7)

### Example 7

The Change in Cancer Positive Cores is Negatively Correlated with the Change in ADP

The change in percentage of cancer-positive cores from biopsy samples taken at the 0 and 12-month time-points of the 12 month dietary intervention study outlined in Example 1 was correlated with the change in ADP concentration at the same time-points. Percentage of cancer-positive cores was assessed using histopathological techniques, and ADP ratio expressed as

Log₂ ADP, was measured by liquid chromatography-mass spectrometry (LC-MS). The change in cancer-positive cores was highly negatively correlated with the change in ADP, such that an increase in ADP correlated with a decrease in cancer-positive cores (FIG. 8).

### Example 8

The Change in Cancer Positive Cores is Negatively Correlated with the Change in sulfate

The change in percentage of cancer-positive cores from biopsy samples taken at the 0 and 12-month time-points of the 12 month dietary intervention study outlined in Example 1 was correlated with the change in sulfate (S) concentration at the same time-points. Percentage of cancer-positive cores was assessed, and sulfate was measured by standard methods (Metabolon Inc.).

A significant inverse association was observed between the change in the level of sulfate (S) in prostate tissue and the percentage of biopsy cores that are positive for cancer (%) (regression analysis shown below), such that an increase in sulfate correlated with a decrease in cancer-positive cores. Sulfate is likely to be derived from the metabolism of S-methylcysteine sulfoxide as shown by Waring et al. (2003), as above.

Regression Analysis: % versus S The regression equation is %=0.897-0.7054 S

S=7.62643 R-Sq=11.4% R-Sq(adj)=9.4%

Analysis of Variance

| Source | DF | SS | MS | F | P |
|---|---|---|---|---|---|
| Regression | 1 | 342.94 | 342.945 | 5.90 | 0.019 |
| Error | 46 | 2675.47 | 58.162 | | |
| Total | 47 | 3018.42 | | | |

This data is represented graphically in FIG. 9

### Example 9

SMCSO Leads to Reduction in Growth of Tumour Cell Line

Cell viability of non-cancerous prostate cells (e.g. PNT1A) and cancerous prostate cells (e.g. DU145 and VCap) are assessed by the WST-1 cell proliferation kit (Roche Applied Science). Cells are seeded in 96-well culture plates and allowed to adhere to the plate surface for 36 h before being exposed to various concentrations of SMCSO (0-200 mM) for 24 h in six replicates. WST-1 (4-[3-(4-Iodophenyl)-2-(4-nitrophenyl)-2H-5-tetrazolio]-1, 3-benzene disulfonate) reagent is then added to each well and incubated for 40 min in a humidified atmosphere (37° C., 5% CO₂). Formazan dye produced by metabolically active cells is measured at 450 nm by microplate ELISA reader (ELx808, Ultra Microplate Reader; BIO-TEK Instruments, Inc., Winooski, Vt.).

### Example 10

Short Duration Diet Intervention Study in Pre-Biopsy Patients

A two arm parallel un-blinded short-term intervention with an enhanced dose of broccoli soups was undertaken. The study recruited men who had been diagnosed or were under investigation for prostate cancer and required a transperineal template biopsy as part of their standard clinical care. The study did not require the participants to have any additional clinical procedures except that during routine sampling separate tissue cores were taken from the prostate and adjacent adipose tissue. Participants in one arm consumed three portions of broccoli and stilton soup per week for at least four weeks prior to their transperineal template biopsy, whereas participants randomised to the other arm did not have any diet intervention prior to biopsy.

Primary Aim

a) To determine whether a broccoli intervention (≥4 weeks, up to 6 weeks) would result in a difference in tissue sulfate levels in men scheduled for prostate biopsies.

Secondary Aims

a) To determine whether a broccoli intervention (≥4 weeks, up to 6 weeks) would result in a difference in tissue ADP levels.

b) To determine whether the diet-induced differences observed in tissue are specific to the prostate gland.

c) To determine whether the diet-induced differences observed in tissue are correlated with differences in urine.

Study Population

This study recruited men (n=18) aged 18-80 years with a BMI between 19.5 and 35 kg/m² with a hospital referral for transperineal template biopsy.

Outline of Study Design

The study outline is presented in FIG. 10. Patients recruited onto this study were randomly allocated to one of two arms in which they were required to consume three portions of broccoli and stilton soup per week for at least four weeks prior to their biopsy (i) or to follow their normal diet until the operation date (ii). The broccoli and stilton soups contained glucoraphanin-enriched Beneforte® broccoli. The study was not blinded and involved tissue, blood and urine collection on the same day that patients were scheduled for their diagnostic biopsy. Their habitual intake of cruciferous vegetables and dietary supplements was assessed.

A human intervention study was undertaken to extend the preliminary findings described herein by exploring whether a short-term intervention with a higher consumption of broccoli induced accumulation of sulphate and ADP in prostate tissue. This provided further information concerning how it is possible to deplete prostate tissue of ATP as a means to reduce risk of cancer progression.

Diagnostic Prostate Biopsy as an Opportunity for a Short Dietary Intervention

Conventional trans-rectal ultrasound scan (TRUS) and prostate biopsy is indicated for investigation of men with a raised prostate-specific antigen (PSA) level and/or digital rectal examination (DRE) suspicious for prostate cancer. Current guidelines from the National Institute of Clinical Excellence (NICE) recommend the use of transperineal template biopsies (TPB) of the prostate for those patients in whom cancer is still suspected but who have had a negative or inconclusive TRUS biopsy (Takenaka, A., et al., A prospective randomized comparison of diagnostic efficacy between transperineal and transrectal 12-core prostate biopsy. Prostate Cancer and Prostatic Diseases, 2008. 11(2): p. 134-138; Hara, R., et al., Optimal approach for prostate cancer detection as initial biopsy: prospective randomized study comparing transperineal versus transrectal systematic 12-core biopsy. Urology, 2008. 71(2): p. 191-5). It is also considered in men on active surveillance, to map the prostate prior to focal therapy or as an adjunct to novel imaging modalities. A pre-biopsy, window-of-opportunity dietary intervention study was undertaken in men who had decided in clinic to proceed to template prostate biopsy. This trial differed from standard window-of-opportunity (phase 0) trials because it was carried out before a diagnostic procedure rather than surgery. The study design also avoided any delay in the patients receiving their investigation for a potential prostate cancer. A similar trial design has been recently published (Atwell, L. L., et al., Sulforaphane Bioavailability and Chemopreventive Activity in Women Scheduled for Breast Biopsy. Cancer Prey Res (Phila), 2015. 8(12): p. 1184-91).

Objectives

A two arm parallel un-blinded dietary intervention study was undertaken as described above. Secondary endpoints of the study were to evaluate ADP accumulation and ATP depletion as a consequence of dietary intervention.

Rather than investigate the effect of the broccoli diet on these individual metabolites within prostate tissue, a global approach was taken using various techniques described in the Experimental Methods section below. Urine samples are analysed to obtain additional information on the systemic effects of the dietary intervention. Whether the accumulation of sulphate and ADP is specific to prostate tissue is assessed through the metabolic analyses of biopsies from prostate and non-prostate tissue. The metabolite profiling of prostate tissue obtained from participants not receiving the broccoli diet and separate core biopsies from pelvic adipose tissue establishes whether diet-induced metabolic differences are specific to the prostate or occur in other tissue compartments.

This study design did not require the participants to have any additional clinical procedures; during the TPB procedure, separate cores were taken from the prostate and adjacent adipose tissue.

Study Design

Volunteers who were being scheduled for TPB were identified by urologists. The ‘window of opportunity’ for dietary intervention was the waiting list time between decision to proceed with TPB and the actual operation date. All TPB procedures were carried out by trained urology consultants, and would otherwise form part of the volunteers' routine clinical care for potential diagnosis and staging of prostate cancer.

Nine (9) volunteers were recruited to each arm of the study.

Volunteers were randomised by “Block randomisation” to 1 of 2 arms:

Arm 1—No dietary intervention

Arm 2—Three portions (300 g each) per week of a soup containing glucoraphanin-enriched broccoli (Beneforte®)

Volunteers' consent was requested for additional cores of prostate tissue to be taken during their TPB that would otherwise form part of their routine clinical care. Consent was also sought for 2 cores of adipose tissue to be taken through the template grid used for the transperineal biopsy, but outside of the prostate. The collection of adipose tissue did not require any additional clinical procedures.

In addition to prostate and adipose tissue, blood and urine samples were collected at the time of TPB. All study samples were used for global and targeted metabolite analyses. A specific dietary questionnaire was carried out at the end of the intervention to establish the volunteers' habitual intake of cruciferous vegetables.

Recruitment Policy A fixed 6-month period from the start of the study was dedicated in order to allow recruitment of 18 volunteers and their follow up to the time of biopsy. Men aged 18 to 80 years and with a BMI of 19.5 to 35 kg/m2 who have agreed to undergo a template biopsy of the prostate were identified and recruited by consultant urologists. The standard waiting list time between a referral for TPB and the actual procedure of 6-8 weeks allowed a dietary intervention of 4-6 weeks from time of recruitment.

Screening Criteria

Basic Inclusion Criteria

Males

Scheduled for TPB as part of routine investigation or staging for prostate cancer

Aged 18-80 years

BMI between 19.5 and 35 kg/m2

Smokers and non-smokers

Basic Exclusion Criteria

Those regularly taking 5a-reductase inhibitors or testosterone replacement medicines

Those on warfarin treatment

Those diagnosed with diabetes

Those diagnosed with or suspected to be high-risk for human immunodeficiency virus (HIV) and/or hepatitis

Those allergic to any of the ingredients of the broccoli and stilton soups

Those taking dietary supplements or herbal remedies which may affect the study outcome. Please note that some supplements may not affect the study and this will be assessed on an individual basis

Those that are unable to understand English or give informed consent

Parallel participation in another research project that involves dietary intervention

Any person related to or living with any member of the study team

Volunteers were provided with a validated Cruciferous Vegetable Food Frequency Questionnaire (CVFFQ) to be completed after the biopsy and returned to the study team using a pre-paid envelope. The habitual consumption of cruciferous vegetables in each group was measured, as participants may already consume high levels of these vegetables as part of their normal diet. CVFFQ is a 6-page, 79-item questionnaire that has been designed and validated by the University of Arizona Health Sciences and assesses cruciferous vegetable intake over the previous 12-month period (Thomson, C. A., et al., Cruciferous vegetable intake questionnaire improves cruciferous vegetable intake estimates. J Am Diet Assoc, 2007. 107(4): p. 631-43). Volunteers were asked to use the CVFFQ to review the time period between the biopsy referral and the actual procedure.

On the day of the biopsy, volunteers were also asked to complete a health questionnaire detailing their past and current medical history and record all regular prescribed and over-the-counter medications.

With informed, signed and written tissue banking consent from all volunteers as detailed above, study samples (blood, urine and tissue biopsies) were collected at the hospital and transported to the study centre to be stored until analysis.

Tissue Biopsy Samples

TPB of the prostate is a useful diagnostic tool in diagnosing and staging prostate cancer. It is performed under general anaesthetic with intra-vascular aminoglycoside antibiotic prophylaxis. The patient is positioned flat on their back (supine) with their legs supported in stirrups and the skin cleaned with an appropriate antiseptic solution. An ultrasound probe is inserted into the rectum and allows direct visualisation of the prostate throughout the procedure for accurate targeting. A specially designed grid is attached to the ultrasound probe and placed flat against the skin of the perineum. Holes in the grid are spaced at 5 mm intervals and allow systematic sampling of all zones of the prostate, while taking care to avoid the urethra. An average of 25 tissue cores is taken, but may vary depending on prostate gland volume.

An additional 8 needle cores of prostate issue were taken through the template grid for study purposes. One core was transferred immediately to methanol for metabolomic analysis, 5 cores were snap frozen in dry ice and stored at −80° C. for target metabolomics, oncogene and protein expression analyses, and 2 cores were placed in RNAlater for RNA extraction.

At the time of template biopsy, 2 cores of adipose tissue from ischio-rectal (pelvic) fat were also taken to act as a control sample. These were taken through the template grid in order to avoid a separate operation site. Both cores were taken at the start of the procedure and using trans-rectal ultrasound to ensure no prostate tissue was involved in the core, before being appropriately processed for subsequent analyses. The collection of additional tissue biopsy cores for this research project was discussed with the consultant urologist and will not affect patients' clinical care. The proposed sample collection is not expected to increase the risks of any associated complications. The prostate biopsies were taken from the same zones as routinely sampled during TPB and the peri-prostatic adipose tissue samples avoided the need for a separate operation site. Intravenous antibiotic prophylaxis was given as standard to reduce the risk of wound infection.

Blood Sample

During routine venous cannulation prior to administration of general anaesthetic (standard procedure for template biopsy), a 5 mL blood sample was collected for genotyping. The blood was mixed with an anti-coagulant (EDTA), divided into aliquots and placed on dry ice until storage at -80° C. until required.

Urine Sample

Prior to the prostate biopsy a first-pass urine sample (20-30m1) was collected following digital rectal examination (DRE) for metabolomic analysis. The sample was divided into aliquots, placed and transported on dry ice and stored at -80° C. until required.

At the end of the study, the remaining blood and urine samples are transferred to a Human Tissue Bank to help future research studies (participants gave written consent for tissue banking). Any further analysis is carried out in full compliance with ethical requirements.

Post-Intervention Follow Up

Volunteers were not required to attend any further clinical follow up as part of the study. Their histology results, including both standard and study cores, were reviewed by the clinician responsible and discussed in a multi-disciplinary team meeting if cancer was detected. All ongoing care was determined as per standard urology treatment.

Study Diet

Volunteers were randomly allocated to either no intervention or consumption of 3 portions of Beneforte® (glucoraphanin-enriched) broccoli and stilton soup per week as part of their normal diet for a minimum of 4 weeks prior to their prostate biopsy. One portion of the

Beneforte broccoli and stilton soup provided 6.4 mmoles SMCSO. As a day-case, elective procedure the waiting time between referral and the actual TPB procedure is currently expected to be 6-8 weeks. Volunteers were asked to continue the soup intervention until the day of the biopsy in case the clinical procedure was delayed. The maximum waiting list duration set by the UK government is 18 weeks, but as an investigation for potential cancer TPBs were prioritised by the hospital.

The Beneforte® broccoli variety was developed by conventional breeding and has the same appearance and flavour as standard broccoli. The broccoli and stilton soups were prepared by Bakkavor®, a leading international producer of freshly prepared foods. Ingredient declarations, nutritional information and allergy statements for the soups were provided from the producer. Bakkavor made frozen soups and then delivered them to the study site. Once delivered, soups were stored in a dedicated freezer. Volunteers were informed on how to consume the soups.

Monitoring of Diet and Compliance

Volunteers randomised to the intervention arm were asked to fill in a soup record sheet during the pre-biopsy intervention period, recording each time they ate their soups. This was intended to aid compliance with the dietary interventions and also to be used in conjunction with the Arizona cruciferous vegetable intake questionnaire. Volunteers were also asked to return the record sheets with the lids of empty soup containers, which were counted by members of the study team to monitor compliance.

Experimental Methods

Prostate Biopsy sampling and processing

8 prostate biopsies and 2 adipose tissue biopsies were collected from each volunteer through a TPB procedure:

Two Cores for Global Metabolomics:

Biopsy samples (1 prostate core, 1 adipose core) were deposited individually into pre-labelled sample vials containing room temperature extraction solvent (methanol). Following incubation at room temperature (up to 48 hours), biopsy samples were removed from the extraction solvent. The removed prostate core was used for histological analysis; instead the adipose core was disposed of by incineration. Histological analysis of prostate biopsies was carried out as part of normal diagnostic procedure for patients under investigation for prostate cancer.

The vials containing extracted metabolites in methanol were stored at -80° C. until required for analysis.

Six Cores for Target Analyses

Biopsy cores (5 prostate cores, 1 adipose core) were placed individually into pre-labelled vials and transported in dry ice until they were stored at -80° C. until required for analysis.

Two Cores for Gene Expression Analyses

Two biopsy cores were immersed in RNAlater solution at the point of collection, and subsequently snap frozen in liquid nitrogen before being stored at −80° C.

Blood Sampling and Processing

A single blood sample (5 ml) was collected by a clinician in the operating theatre before starting the biopsy procedure. This was divided into aliquots, transported on dry ice and stored at −80° C. until required for genotyping.

Urine Sampling and Processing

A first-pass urine sample was collected following prostate massage immediately before the patient underwent the prostate biopsy procedure. This was divided into aliquots, transported on dry ice and stored at -80° C. until required for global and targeted metabolomic analysis.

Metabolite Profiling

The metabolomic analysis of tissue and urine samples is carried out by a US company called Metabolon®. Metabolon® is a service and diagnostic products company with the ability to identify and produce a profile of up to 350 known metabolites using standard metabolomics techniques including liquid chromatography/mass spectrometry (LC/MS) and gas chromatography/mass spectrometry (GC/MS) (Sreekumar, A., et al., Metabolomic profiles delineate potential role for sarcosine in prostate cancer progression. Nature, 2009. 457(7231): p. 910-4).

Tissue and Urine Samples are Sent for Analysis at:

Metabolon Sample Acceptance,

800 Capitola Drive, Suite 1

Durham, N.C. 27713, USA.

Briefly, the sample preparation process is carried out using the automated MicroLab STAR® system from Hamilton Company. Standards are added prior to the first step in the extraction process for quality control purposes. Samples are prepared using a series of organic and aqueous extractions to remove the protein fraction while allowing maximum recovery of small molecules. The resulting extract is then divided into two fractions; one for analysis by LC and one for analysis by GC. Samples are placed briefly on a TurboVap® (Zymark) to remove the organic solvent. Each sample is then frozen and dried under vacuum and thus prepared for the appropriate instrument, either LC/MS or GC/MS.

Metabolon® provides detailed reports on the difference in levels of sulphate, ADP and phosphate as well as a much wider range of metabolites following dietary intervention. The Metabolon® platform can identify sulphate with very high confidence levels, even without an internal standard. Sulphate has a unique mass defect and isotope pattern which are highly characteristic of the sulphur atom, ensuring fragmentation is accurate.

Sulphate and other relevant metabolites are also measured in tissue and urine samples by chromatography methods following standard operating procedures. Sulphate quantification is performed by liquid chromatography/mass spectrometry using an external standard calibration curve.

Target analyses to determine ADP/ATP levels in tissue samples are carried out using commercially available assay kits.

Statistical Analysis

Sample Size Calculation

There are no data (from previous literature) reporting the effect of a diet rich in broccoli on sulphate levels within prostate and at the systemic level. The sample size for the proposed study has been estimated taking into consideration the preliminary data obtained from the metabolomic analysis of samples collected as part of the 12-month dietary intervention study (e.g. Example 1). The metabolite profiling of prostate tissue from patients (n=15) randomised to a 12-month broccoli intervention has shown a significant accumulation of sulphate in prostate tissue after the intervention period compared to baseline (Example 4).

To detect a difference of 1.735 (normalised ion count) at a 5% significance level with 90% power and assuming a within group standard deviation of 1.056 (normalised ion count) requires a sample size of 9 individuals in each group (total 18). These sample sizes were calculated for a two-group study design (broccoli intervention vs no intervention) assuming a two-sided comparison (i.e. to detect a difference rather than a higher level).

Data Analysis

The statistical analysis for data generated from this study is performed by using different methods.

To determine if the dietary intervention affects sulphate levels, a Welch's t-test (or unequal variance test) is used for data obtained from the two groups (broccoli intervention vs no intervention).

One of the secondary aims of this study is to determine whether metabolic differences occurring in response to diet within prostate tissue are correlated with metabolite levels in urine.

The association between the metabolic differences in prostate and those measured in urine is assessed using an appropriate correlation test.

The analysis uses ANCOVA to test for the effect of the diet whilst taking account of the glutathione S-transferase Mu 1 (GSTM1) genotype or other relevant genotypes. Genotype is included as an explanatory variable in this analysis.

### Example 11

Accumulation of SMCSO and ADP in Human Prostate Tissue In Vivo

Following the short dietary intervention study described in Example 10, the SMCSO level in prostate biopsy tissue was compared between the volunteers in Arm 1 (no dietary intervention) and Arm 2 (dietary intervention).

SMCSO was measured by following the method described by Bernaert and colleagues (Bernaert, N., et al., Influence of cultivar and harvest time on the amounts of isoalliin and methiin in leek (Allium ampeloprasum var. porrum). J Agric Food Chem, 2012. 60(44): p. 10910-9). Samples were extracted on ice by adding 50% trichloroacetic acid (TCA) and vortexed for 30 seconds. After centrifugation, the supernatant was diluted with 0.1% formic acid in water, and analysed by LC-MS/MS using an Agilent 6490 mass spectrometer with a photodiode array detector. The LC-MS/MS was set on a flow rate of 0.3 mL/min. The column used for the analysis was an Agilent SB-AQ 1.8 uM (100×2.1mm) C18 column with an Agilent Zorbax guard column. The column temperature and auto sampler were maintained at 20° C. and 4° C. respectively. Fragment separation and detection was conducted using MRM mode detection which increases the lower detection limit.

FIG. 11 shows an increased accumulation of SMCSO in prostate biopsy tissue on average from the volunteers in the dietary intervention arm compared with those in the non dietary intervention arm. This indicates that SMCSO can accumulate in the prostate in vivo when an SMCSO-enriched diet is followed for a relatively short (>4 weeks) timeframe.

The data presented in FIG. 12 shows an increase in ADP in volunteers in the dietary intervention arm compared to those in the non dietary intervention arm.

1-21. (canceled)

22. A composition comprising S-methylcysteine sulfoxide, an analogue, derivative, metabolite or pharmaceutically acceptable salt thereof, for use in the treatment and/or prevention of prostate cancer.

23. The composition as claimed in claim 22 wherein the prostate cancer is early-stage prostate cancer.

24. The composition as claimed in claim 22 further comprising glucoraphanin, an analogue, derivative or metabolite thereof.

25. The composition as claimed in claim 22 wherein said composition is a pharmaceutical composition.

26. The composition as claimed in claim 25 further comprising one or more pharmaceutically acceptable carriers or excipients.

27. The composition as claimed in claim 22 wherein said composition is a food composition.

28. The composition as claimed claim 27 wherein the food composition is a soup.

29. The composition as claimed in claim 27 comprising a tomato species or a compound derived from a tomato species.

30. The composition as claimed in claim 27 wherein the food composition is a food supplement.

31. The composition as claimed in claims 27, wherein the S-methylcysteine sulfoxide, analogue, derivative or metabolite thereof is added as an additive.

32. The composition as claimed in claim 22 wherein said composition is in the form of tablets, capsules, ovules, elixirs, solutions or suspensions.

33. The composition as claimed in claim 22 wherein said composition is provided in a dosage form to achieve a reduction in the number of needle core biopsies that are positive for prostate cancer.

34. The composition according to claim 22 wherein said composition provides a dose of the S-methylcysteine sulfoxide or analogue, derivative or metabolite thereof in an amount in a range of from 3 to >10 mmoles.

35. The composition as claimed in claim 34 wherein the dose provided is an amount in a range of from 3 to about 50 mmoles.

36. The composition as claimed in claim 22 wherein said composition is for administration once per week.

37. The composition as claimed in claim 36 wherein said composition is for administration to provide with a weekly dose in the range of 3 to about 50 mmoles of S-methylcysteine sulfoxide.

38. The composition as claimed in claim 22 for use in combination with at least one therapy selected from the group consisting of: chemotherapy, hormone therapy ef and radiotherapy in the treatment of prostate cancer.

39. A use of a composition comprising S-methylcysteine sulfoxide, an analogue, derivative, metabolite or pharmaceutically acceptable salt thereof for the manufacture of a medicament for use in the treatment and/or prevention of prostate cancer.

40. A prostrate cancer treatment and prevention method comprising administering a composition comprising a therapeutically effective amount of S-methylcysteine sulfoxide, an analogue, derivative, metabolite or pharmaceutically-acceptable salt thereof.

41. A composition as claimed in claim 22 wherein the S-methylcysteine sulfoxide, an analogue, derivative or metabolite thereof and/or glucoraphanin are derived from plants of the Brassicales or Asparagales orders.
